(19) United States
(12) Patent Application Publication
(10) Pub. No.: US 2004/0127438 A1
(43) Pub. Date: Jul. 1, 2004

(54) Biological materials and uses thereof

(76) Inventor: Anthony Robert Milnes Coates, Ore (GB)

Correspondence Address: SUGHRUE MION, PLLC, 2100 PENNSYLVANIA AVENUE, N.W., SUITE 800, WASHINGTON, DC, 20037, US

(21) Appl. No.: 10/416,884

(22) PCT Filed: Nov. 16, 2001
(86) PCT No.: PCT/GB01/05084

(30) Foreign Application Priority Data
Nov. 17, 2000 (GB) 0028122.0

(51) Int. Cl. A61K 38/17; A61K 48/00

(52) U.S. Cl. 514/44; 514/12

(57) ABSTRACT

The invention relates to the use of cpn 10 from Mycobacterium tuberculosis, or functionally equivalent molecules of fragments thereof, in the prevention and/or treatment of allergic conditions such as asthma, rhinitis/hay fever, eczema and anaphylaxis, which conditions are associated with an underlying Th2 cell over-reactivity, or cancerous conditions.

The present invention relates to the use of an approximately 10 kDa polypeptide (or its encoding nucleic acid molecule) or functionally equivalent molecules or fragments thereof from Mycobacterium tuberculosis or related prokaryotes in the treatment of cancer, allergic disorders or conditions of immunoactivation, particularly asthma, and/or conditions typified by a T helper lymphocyte 2 (Th2)-type immune response and/or conditions associated with eosinophilia and methods of stimulating the production of immune response mediators, e.g. cytokines, in vitro or in vivo.

Autoimmunity reflects the loss of tolerance to “self” resulting in inappropriate destruction of normal cells or tissue. In many conditions, autoantibodies are found, but may reflect an effect rather than cause of a disease. In some diseases however autoantibodies are the first, major, or only detectable abnormality. one class of molecules which is implicated in this respect are the chaperonins which are highly immunogenic. Chaperonins belong to a group of proteins called molecular chaperones which bind non-native proteins and assist them, in an ATP-dependent catalytic process, to fold into the correct three-dimensional form required for a functional protein.

Chaperonins are believed to stimulate the immune system at many levels simultaneously, including monocytes, macrophages, fibroblast-like cells, perhaps other types of cells, and T cells. The immune defences in mammals may be divided into the “innate” and “adaptive” defences. Those which are already in place, such as phagocytes, natural killer cells and complement are considered innate. On challenge, adaptive immunity is activated in the form of B and T lymphocytes. Chaperonins are known to act directly on the innate defence mechanisms, particularly on phagocytes. They also stimulate a powerful adaptive immune response, namely the production of antibody and the stimulation of T lymphocytes which in some cases may be protective. Notably they induce cytokine secretion which is thought to be important for host defences. In some cases however it is believed that the presence of chaperonins may be damaging to the host.

Ragno et al (1996) Clin Exp Immunol 103: 384-390 showed that an aqueous solution of a 10-kD heat shock protein (hsp10) from Mycobacterium tuberculosis delayed the onset and severity of adjuvant-induced arthritis (AA) in rats. AA is a model of autoimmune diseases, which are associated with an underlying over-reactivity of Th-1 cells. There is no suggestion that hsp10 could find utility in the treatment of allergic conditions such as asthma, rhinitis/hay fever, eczema, analphylaxis and the like, which, in contrast to autoimmune diseases (Th1 over-reacting) are associated with an underlying over-reactivity of Th-2 cells.

Chaperonins' role in autoimmune disease is controversial. Although infection/immunity with chaperonin-containing organisms is universal, and healthy people have T cell responses to self-chaperonins, including the production of chaperonin-specific antibodies, classical autoimmune disease is quite uncommon. So the presence of immune reactions to chaperonins may be incidental and unimportant.

The theory of molecular mimicry however suggests the involvement of chaperonins in autoimmune disease and is based on the high level of amino acid sequence conservation between chaperonins of microbial and mammalian origin. The theory proposes that during infection with a wide range of microbes, chaperonin epitopes that are shared between microbes and mammals stimulate T lymphocytes. According to this theory a high level of chaperonin presentation of shared chaperonin epitopes breaks tolerance to self-chaperonins and autoimmune disease develops.

Chaperonins obtained from tumours have been found to result in necrotic effects on those tumours. It is suggested that this may be achieved through enhancing immunological recognition of tumour antigens although the mechanism of this is not known. It therefore appears that chaperonins induce protective adaptive immunity against bacterial infection and cancer.

Allergic reactions, such as asthma, concern proportionally inappropriate or misdirected immune responses. The prevalence of asthma for example is increasing and effective therapies for treating all cases have not yet been found. Current treatment often uses immunosuppressive glucocorticosteroids, beta agonists, cromoglycate, leukotriene modifiers etc. which have numerous side-effects.

In such allergic reactions, high IgE levels occur and T helper lymphocyte-2 (Th2) immune responses predominate over Th1 responses resulting in an inflammatory response. Th1 responses are thought to be mainly protective against microbial infection and are promoted by cytokines, particularly interleukin-12 (IL12), IL-2 and interferon-γ. In contrast, Th2 responses, in the appropriate genetic background, are associated with harmful allergic tissue damage.

However, it has been suggested that in other conditions such as autoimmune disorders, e.g. adjuvant arthritis, overactive Th1 responses are causal of the disorder. Conversion of Th1 to Th2 or Th2 to Th1 responses may therefore have utility in treating the above described disorders.

Whilst it has been known that bacteria such as L. monocytogenes, M. bovis and M. tuberculosis can convert Th2 to Th1 responses, the molecules which is (are) responsible for this conversion have not been identified.

Suggestions in the art have however implicated a heat shock protein, hsp65, from M. leprae which is able to induce Th1 responses (Lowrie et al., 1999, Nature, 400, p269-271; Bonato et al., 1998, Infect. Immun., 66, p169-175). The homologue, hsp65 from M. tuberculosis, has the ability to stimulate human monocytes to synthesize pro-inflammatory cytokines and activate monocytes and human vascular endothelial cells (Friedland et al., 1993, Clin. Exp. Immunol., 91, p5862; Peetermans et al., 1995, Infect. Immun., 63, p3454-3458; Verdegaal, et al., 1996, J. Immunol., 157, p369-376).

Surprisingly it has now been found that another protein is able to affect the immunity of an individual and can be used for treating or preventing conditions such as cancer, allergic conditions such as asthma and/or conditions typified by a Th2-type immune response and/or conditions associated with eosinophilia.

This protein from Mycobacterium tuberculosis has surprisingly been found to have advantageous properties in treating the aforementioned conditions. This 10 kDa molecule is termed Mycobacterium tuberculosis chaperonin 10 (Mtcpn 10).

Cpn 10 which is a chaperonin and a heat shock protein is under discrete transcriptional control to the molecules cpn 60.1 and 60.2.

The invention therefore provides molecules such as Mycobacterium cpn 10 which have enhanced properties in treating or preventing various disorders such as cancer, allergic reactions and/or conditions typified by a Th2-type immune response and/or conditions associated with eosinophilia.

Therapeutic and/or prophylactic applications may be achieved using nucleic acid molecules or peptides/proteins, as will be described in more detail hereinafter.

Thus, in a first aspect the present invention provides a pharmaceutical composition comprising a nucleic acid molecule comprising

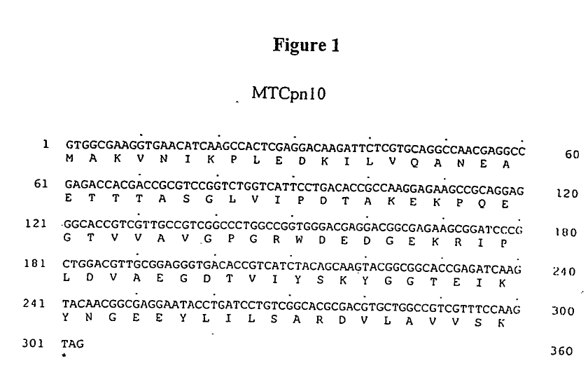

(i) the nucleotide sequence of FIG. 1, or

(ii) a sequence which has more than 66%, e.g. 70 or 75%, preferably more than 80%, e.g. more than 90 or 950% identity to sequence (i) (according to the test described hereinafter) or a sequence which hybridizes to sequence (i) under conditions of 2×SSC, 65° C. (wherein SCC=0.15M NaCl, 0.015M sodium citrate, pH 7.2) which encodes a functionally equivalent protein to the sequence encoded by the nucleotide sequence of FIG. 1, or (iii) a fragment of any sequence (i) or (ii) encoding a functionally equivalent protein fragment; and a pharmaceutically acceptable excipient, diluent or carrier.

As mentioned above, therapeutic and/or prophylactic effects may be achieved using nucleic acid molecules or peptide/protein molecules. Thus in a further aspect the present invention provides a pharmaceutical composition comprising a polypeptide comprising

(i) the amino acid sequence of FIG. 1, or

(ii) a sequence which has more than 60%, e.g. 65 or 70%, preferably more than 80%, e.g. more than 90 or 95% homology to sequence (i) (according to the test described hereinafter) which provides a functionally equivalent protein, or

(iii) a functionally equivalent fragment of any sequence (i) or (ii); and a pharmaceutically acceptable excipient, diluent or carrier.

“Nucleic acid molecules” according to the invention may be single or double stranded DNA, cDNA or RNA, preferably DNA. Derivatives of nucleotide sequences capable of encoding functionally-equivalent polypeptides may be obtained by using conventional methods well known in the art.

Nucleic acid molecules for use in the invention may consist only of sequences derived from FIG. 1 (or related functionally equivalent sequences), or may comprise additional sequences, such as structural or functional sequences, e.g. sequences which control transcription and/or expression (particularly in mammalian cells), or sequences which comprise the sequence for an additional protein moiety which may form a fusion protein which may have specific properties e.g. act as a secretary signal. Thus for example the sequence may be in the form of a vector containing the nucleic acid molecules described herein. Suitable vectors include plasmids and viruses.

“Polypeptides” as referred to herein includes both full-length protein and shorter length peptide sequences, e.g. protein fragments as described herein. Such polypeptides may be prepared by any convenient means, e.g. by isolation from the source prokaryote or by recombinant means by expression of the appropriate nucleic acid molecule in a host cell operatively linked to an expression control sequence, or a recombinant DNA cloning vehicle or vector containing such a recombinant DNA molecule or by chemical or biochemical synthesis (ex vivo).

“Sequence identity” as referred to herein in connection with nucleotide sequences refers to the value obtained when assessed using ClustalW (Thompson et al., 1994, Nucl. Acids Res., 22, p4673-4680) with the following parameters:

Pairwise alignment parameters—Method: accurate,

Matrix: IUB, Gap open penalty: 15.00, Gap extension penalty: 6.66;

Multiple alignment parameters—Matrix: IUB, Gap open penalty: 15.00, % identity for delay: 30, Negative matrix: no, Gap extension penalty: 6.66, DNA transitions weighting: 0.5.

In connection with amino acid sequences, “sequence identity” refers to sequences which have the stated value when assessed using ClustalW (Thompson et al., 1994, supra) with the following parameters: Pairwise alignment parameters—Method: accurate,

Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

“Functionally equivalent” proteins or protein fragments refers to proteins or fragments related to, or derived from the amino acid sequence of FIG. 1, where the amino acid sequence has been modified by single or multiple amino acid (e.g. at 1 to 50, e.g. 10 to 30, preferably 1 to 5 bases) substitution, addition and/or deletion but which nonetheless retains functional activity, e.g. suppresses ovalbumin-induced eosinophilia, for example reducing eosinophil numbers to the extent of more than 10%, e.g. more than 25%, particularly preferably more than 50% and/or an increase in the production of specific cytokines such as interleukin-1β (IL-1β), IL-2, IL-6, IL-8, IL-10, IL-12, IL-12 receptor, tumour necrosis factor a (TNFα) interferon-γ and granulocyte-macrophage-colony stimulating factor (GM-CSF) e.g. a more than 10 fold, preferably more than 100 fold increase over normal levels and/or stimulation of Th1 responses.

Within the meaning of “addition” variants are included amino and/or carboxyl terminal fusion proteins or polypeptides, comprising an additional protein or polypeptide fused to the polypeptide sequence.

Particularly preferred are naturally occurring equivalents such as biological variations, e.g. allelic, geographical or allotypic variants and derivatives prepared using known techniques. For example, functionally-equivalent proteins or fragments may be prepared either by chemical peptide synthesis or in recombinant form using the known techniques of site-directed mutagenesis, random mutagenesis, or enzymatic cleavage and/or ligation of nucleic acids.

The invention is particularly directed to homologues and related molecules from different prokaryotes, e.g. from bacterial genera, species or strains, particularly from the genus Mycobacterium, e.g. homologues from the Mycobacterium tuberculosis complex which includes M. tuberculosis, M. bovis and M. africanum. Such sequences may themselves be modified, particularly derivatized providing they still retain functionality.

Derivatives of the proteins may be prepared by post-synthesis/isolation modification or by modification during synthesis, e.g. using modified residues or expression of modified nucleic acid molecules, where appropriate.

Functionally-equivalent fragments according to the invention may be made by truncation, e.g. by removal of a peptide from the N and/or C-terminal ends or by selection of an appropriate active domain region, e.g. an epitopic region which retains its functionality. Such fragments may be derived from the sequence of FIG. 1 or may be derived from a functionally equivalent protein to that disclosed in FIG. 1.

It will be appreciated that where functional fragments are selected they may not exhibit all functions attributed to the source molecules. Thus functionally equivalent proteins or fragments refers to retention of relevant functional properties such that the fragment retains utility according to the invention, e.g. reduces eosinophilia, increases the production of specific cytokines and/or stimulates the Th1 immune response, as mentioned above. Preferably the fragments are between 6 and 99 residues in length, e.g. 15 to 99 residues, preferably 6 to 30, 10 to 25, 15 to 50 or 15 to 30 residues. Particularly preferred fragments of the sequence shown in FIG. 1 are those derived from or consisting of the following residues:—

1-25

1-58

25-99

51-99

75-99

Functionally equivalent nucleic acid sequences/fragments compared to the sequence recited in FIG. 1 are also used in compositions of the invention. These sequences are defined with reference to the functionally equivalent protein/peptides (as defined above) which they encode.

“Hybridisation” as used herein refers to those sequences which bind under non-stringent conditions (6×SSC/50% formamide at room temperature) and washed under conditions of high stringency e.g. 2×SSC, 65° C. (where SSC=0.15M NaCl, 0.015M sodium citrate, pH 7.2).

“Pharmaceutically acceptable” as referred to herein refers to ingredients that are compatible with other ingredients of the compositions as well as physiologically acceptable to the recipient.

Pharmaceutical compositions according to the invention may be formulated in conventional manner using readily available ingredients. Thus, the active ingredient (ie. the nucleic acid molecule or protein/peptide), may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile packaged powders, and the like.

As mentioned above, compositions may additionally comprise molecules which assist or augment the action of the nucleic acid molecules or polypeptides described hereinbefore, e.g. thalidomide (and analogues thereof), low dose cyclophosphamide, LPS, cytokines, chemokines, CpG oligodeoxynucleotides and other immunomodulators and/or anti-inflammatory agents such as cytokine antagonists or glucocorticosteroids.

Thus for example, the compositions may be used together with active ingredients for specific immunotherapies e.g. in cancer vaccines. Appropriate immunotherapy treatment/vaccine preparations which may include nucleic acid molecules/polypeptides as described herein include subunit vaccines or treatments based on tumour specific antigens or tumour associated antigens or antibody, anti-idiotype antibody or whole cell preparations for vaccination or therapy. When used in therapy or vaccination the nucleic acid molecules or polypeptides described herein may provide (or encode) an antigen resulting in a specific immune response directed to that antigen and/or may result in a general and nonspecific immune response. In the latter case in which compositions containing other active ingredients are used, the nucleic acid molecules/polypeptides described herein act as adjuvants and may be used for this purpose.

Preventative or therapeutic preparations may be formulated to include one or more suitable adjuvants, e.g. Incomplete Freund's Adjuvant, BCG, Montanide, aluminium hydroxide, saponin, quil A, or more purified forms thereof, muramyl dipeptide, mineral or vegetable oils, Novasome or non-ionic block co-polymers or DEAE dextran, in the presence of one or more pharmaceutically acceptable carriers or diluents. Suitable carriers include liquid media such as saline solution.

Examples of suitable carriers, excipients, and diluents are lactose, dextrose, sucrose, sorbitol, mannitol, starches, gum acacia, calcium phosphate, aglinates, tragacanth, gelatin, calcium silicate, microcrystalline cellulose, polyvinylpyrrolidone, cellulose, water syrup, water, water/ethanol, water/glycol, water/polyethylene glycol, propylene glycol, methyl cellulose, methylhydroxybenzoates, propyl hydroxybenzoates, talc, magnesium stearate, mineral oil or fatty substances such as hard fat or suitable mixtures thereof. The compositions may additionally include lubricating agents, wetting agents, emulsifying agents, suspending agents, preserving agents, sweetening agents, flavouring agents, and the like. The compositions of the invention may be formulated so as to provide quick, sustained or delayed release of the active ingredient after administration to the patient by employing procedures well known in the art.

Compositions may be in an appropriate dosage form, for example as an emulsion or in liposomes, niosomes, microspheres, nanoparticles or the like.

If required, the compositions may also contain targeting moieties attached to the active ingredient, e.g. a ligand which binds specifically and selectively to an endogenous receptor to allow targeting to a particular cell type or location, such as targeting to lymphocytes, monocytes, macrophages, endothelial cells, epithelial cells, blood cells, erythrocytes, platelets, eosinophils, neutrophils, natural killer cells, dendritic cells, brain cells, heart cells, lung cells, islet cells, kidney cells, cancer cells, hormonal gland cells, skin, bone, joints, bone marrow, gastric mucosa, lymph nodes, Peyers patches, the omentum and other immunological tissues.

The above described compositions have utility in the treatment or prophylaxis of cancer, allergic reactions and/or conditions typified by a Th2-type immune response and/or conditions associated with eosinophilia.

Thus in a further aspect the present invention provides pharmaceutical compositions as described herein for use as a medicament, preferably as an immunosuppressant, e.g. for use in treating or preventing cancer, allergic reactions and/or conditions typified by a Th2-type immune response and/or conditions associated with eosinophilia.

Alternatively viewed, the present invention provides a method of treating or preventing cancer, allergic responses and/or conditions typified by a Th2 type immune response and/or conditions associated with eosinophilia in a patient wherein said patient is administered a pharmaceutical composition as described hereinbefore.

Furthermore, the present invention provides the use of a nucleic acid molecule comprising

(i) the nucleotide sequence of FIG. 1, or

(ii) a sequence which has more than 66%, e.g. 70 or 75%, preferably more than 80%, e.g. more than 90 or 95% identity to sequence (i) (according to the test described hereinbefore) or a sequence which hybridizes to sequence (i) under conditions of 2×SSC, 65° C. (wherein SCC=0.15M NaCl, 0.015M sodium citrate, pH 7.2) which encodes a functionally equivalent protein to the sequence encoded by the nucleotide sequence of FIG. 1, or (iii) a fragment of sequence (i) or (ii) encoding a functionally equivalent protein fragment; or

a polypeptide comprising

(i) the amino acid sequence of FIG. 1, or

(ii) a sequence which has more than 60%, e.g. 65 or 70%, preferably more than 80%, e.g. more than 90 or 95% homology to sequence (i) (according to the test described hereinbefore) which provides a functionally equivalent protein, or

(iii) a functionally equivalent fragment of sequence (i) or (ii);

in the preparation of a medicament for treating or preventing cancer, allergic responses and/or conditions typified by a Th2-type immune response and/or conditions associated with eosinophilia.

As defined herein “treatment” refers to reducing, alleviating or eliminating one or more symptoms of the condition which is being treated, relative to the symptoms prior to treatment. For example, symptoms which may be affected include eosinophilia, decreased secretion of particular cytokines, a Th2-biased immune response, tumour size (e.g. by halting proliferation, causing differentiation, enhancing or inducing antitumour immune responses or causing some cell death), allergic response, presence of autoantibodies, etc which are treated to achieve the effects particularly as defined in respect of the functional properties of functionally equivalent polypeptides.

“Prevention” of a condition refers to delaying or preventing the onset of a condition or reducing its severity, as assessed by the appearance or extent of one or more symptoms of said condition.

Cancers which may be prevented or treated include malignant and pre-malignant or benign tumours and include carcinomas, sarcomas, glioma, melanoma and Hodgkin's disease, including cancers of the bladder, kidney, pancreas, brain, head and neck, breast, gut, prostate, lung and ovary and leukaemias and lymphomas.

Allergic conditions which may be treated or prevented include eczema, dermatitis, allergic rhinitis, allergic conjunctivitis, allergic airway diseases, hyper-eosinophilic syndrome, contact dermatitis, food allergy, and respiratory diseases characterized by eosinophilic airway inflammation and airway hyperresponsiveness, such as allergic asthma, intrinsic asthma, allergic bronchopulmonary aspergillosis, eosinophilic pneumonia, allergic bronchitis bronchiectasis, occupational asthma, reactive airway disease syndrome, interstitial lung disease, hypereosinophilic syndrome or parasitic lung disease. Preferably however the composition is used for treating asthma. In a further preferred feature, the composition is used for treating conditions in which eosinophilia plays a role, e.g. allergies (as described above, particularly asthma), atopic disorders and pulmonary eosinophilia.

Patients which may be treated include, but are not limited to mammals, particularly primates, domestic animals and livestock. Thus preferred animals for treatment include mice, rats, guinea pigs, cats, dogs, pigs, goats, sheep, horses and particularly preferably, humans.

As mentioned previously, either nucleic acid molecules or polypeptides may be used in the methods of the invention. In instances in which nucleic acid molecules are employed, these are conveniently applied in a form to allow their expression within the patient, thus providing a form of gene therapy. Thus the pharmaceutical compositions described herein containing a nucleic acid molecule may be used in methods of gene therapy.

Thus for example the nucleic acid molecules may be provided in a liposome, micelle or other convenient carrying vehicle which may comprise targeting moieties to allow its targeting to cells of interest.

Alternatively the molecules may be packaged in other, “vehicles” such as viruses, plasmids or cells (particularly transfected species-matched cells) which are all well known in the art for this purpose which allow expression of the resident molecule.

Appropriate techniques for transfection are well known and include electroporation, microinjection, lipofection, adsorption, viral transfection and protoplast fusion.

Administration of compositions of the invention may take place by any of the conventional routes, e.g. by inhalation, nasally, orally, rectally or parenterally, such as by intramuscular, subcutaneous, intraperitoneal or intravenous injection. Treatment or prophylaxis by topical application of a composition, e.g. an ointment, to the skin is also possible. Optionally administration may be performed at intervals, e.g. 2 or more applications, e.g. 2-4 applications at hourly, daily, weekly or monthly intervals, e.g. several times a day, or every 3-5 days, or at fortnightly, monthly or quarterly intervals.

It has been observed in work conducted on the related molecule cpn 60.2 that the route of administration may affect the immune response which is generated. For example when Mtcpn 60.2 is administered intranasally, a Th2 to Th1 shift is stimulated although the reverse effect is observed when administered intraperitoneally. Thus, the route of administration should take into account the disorder to be treated/prevented and thus for example in treating autoinimune disorders, intraperitoneal administration may be appropriate whereas treatment or prevention of particularly allergic disorders may be for example by intranasal administration.

In prophylactic methods of the invention, administration (conveniently orally or by inhalation or subcutaneous or intramuscular injection) is preferably performed at more lengthy intervals, e.g. intervals of 2-12 weeks. For therapeutic purposes, administration (conveniently orally or by inhalation or intravenous injection) is performed 1-4 times in a single day or over 2 days.

The active ingredient in composition of the invention may comprise from about 0.01% to about 99% by weight of the formulation, preferably from about 0.1 to about 50%, for example 10%. The compositions are preferably formulated in a unit dosage form, e.g. with each dosage containing from about 0.01 mg to about Ig of the active ingredient, e.g. 0.05 mg to 0.5 g, for a human, e.g. 1-100 mg.

The precise dosage of the active compound to be administered and the length of the course of treatment will, of course, depend on a number of factors including for example, the age and weight of the patient, the specific condition requiring treatment and its severity, and the route of administration. Generally however, an effective dose may lie in the range of from about 0.1 μg/kg to about 14 mg/kg, preferably 0.1 to 1 mg/kg, e.g. from about 1 mg to 1 g of polypeptide per day, depending on the animal to be treated and the dosage form, taken as a single dose. Thus for example, an appropriate daily dose for an adult may be from 7 μg to 1 g, e.g. 10 mg to 1 g per day, e.g. 25 to 500 mg of the polypeptide per day.

Similar or lower dosages may be used when using nucleic acid molecules described herein, e.g. from about 0.2 ng/kg to about 2.5 mg/kg (e.g. from about 0.2 ng/kg to about 2 ng/kg or about 1.5 ng/kg to about 2.5 mg/kg) such as about 14 ng to about 175 mg for an adult. However, where the nucleic acid molecules are packaged in cells or vectors proportionally higher or lower amounts may be required depending on the extent of non-cpn encoding DNA and sequences which influence the level of expression, e.g. 5 or 10-fold larger amounts, e.g. nucleic acid molecules described herein packaged in a vector may be used at about 1.0 ng/kg to about 12.5 mg/kg.

As mentioned above, the family of polypeptides defined herein and the nucleic acid molecules encoding them stimulate the production of a set of cytokines. This therefore allows the use of these compounds for the express purpose of stimulating production of these cytokines whether or not this occurs in a therapeutic/prophylactic situation. Thus in a further aspect the present invention provides a method of stimulating cytokine production in a cell, wherein said method comprises administration of

a nucleic acid molecule comprising

(i) the nucleotide sequence of FIG. 1, or

(ii) a sequence which has more than 66%, e.g. 70 or 75%, preferably more than 80%, e.g. more than 90 or 95% identity to sequence (i) (according to the test described hereinbefore) or a sequence which hybridizes to sequence (i) under conditions of 2×SSC, 65° C. (wherein SCC=0.15M NaCl, 0.015M sodium citrate, pH 7.2) which encodes a functionally equivalent protein to the sequence encoded by the nucleotide sequence of FIG. 1, or (iii) a fragment of sequence (i) or (ii) encoding a functionally equivalent protein fragment; or

a polypeptide comprising

(i) the amino acid sequence of FIG. 1, or

(ii) a sequence which has more than 60%, e.g. 65 or 70%, preferably more than 80%, e.g. more than 90 or 95% homology to sequence (i) (according to the test described hereinbefore) which provides a functionally equivalent protein, or

(iii) a functionally equivalent fragment of sequence (i) or (ii); to said cell.

Such methods may be performed in vitro, e.g. on cells, tissues or organs outside the body. This methodology may for example be used in research methods to identify the molecule or molecules which react or bind to or are activated via molecules of the invention, e.g. cpn 10 receptor molecules As a corollary to such methods, the stimulation of cytokine production may be used to measure the presence of molecules of the invention.

Thus, in a further aspect the present invention provides a method of assessing the presence or concentration of a polypeptide or peptide of the invention in a sample wherein said sample is applied to a cell and the level of production of one or more cytokines is measured and compared to the level of production of said one or more cytokines in a control sample wherein the increase over control levels provides a correlation to the presence or concentration of said polypeptide or peptide in said sample.

As used herein “control” refers to a sample which does not contain molecules of the invention or moieties which increase production of the cytokine(s) to be measured. Where appropriate, standard curves may be generated using molecules of the invention to allow quantitative assessment to be made of the presence or concentration of said molecules, although qualitative assessments may also be made. This method may furthermore be used to identify molecules of the invention.

Alternatively however, the method of stimulating cytokine production may be performed in vivo to enhance production of particular cytokines. This may have beneficial therapeutic or prophylactic effects and in which case the invention extends to the nucleic acid molecules and polypeptides as described above for use in treating conditions which may be alleviated, overcome or prevented by increasing specific cytokines, and the use of such molecules for the preparation of medicaments for that purpose.

Preferably the cytokines which are increased, e.g. more than 10 or 100 fold relative to normal levels, are selected from the group consisting of IL-1β, IL-2, IL-6, IL-8, IL-10, IL-12, TNFα, interferon-γ and GM-CSF.

Definitions

“AUTOIMMUNE DISEASE”. This term intended to cover those cases where it can be shown that the autoimmune process contributes to the pathogenesis of a disease. Such diseases are typically associated with a Thelper lymphocyte-1 (Th-1) type immune response.

“ALLERGIC CONDITIONS”. This term is intended to cover conditions asociated with a T helper lymphocyte-2 (Th-2) type immune response. In allergic reaction, high IgE levels occur and Th-2 immune responses predominate over Th-1 responses, resulting in inflammatory response. Examples of allergic conditions include the following: asthma, rhinitis/hay fever, eczema and anaphylaxis.

“ADJUVANT”. This term is intended to cover any substance which, when incorporated into or administered simultaneously with antigen, potentiates the immune response.

“Mtcpn10”, “cpn 10”, “hsp10”, and “Pep10” are used interchangeably throughout the specification to refer to the amino acid sequence shown in FIG. 1.

The invention will now be described in more detail by way of the following non-limiting Examples in which:—

FIG. 1 shows the nucleotide and amino acid sequence of cpn 10 from M. tuberculosis;

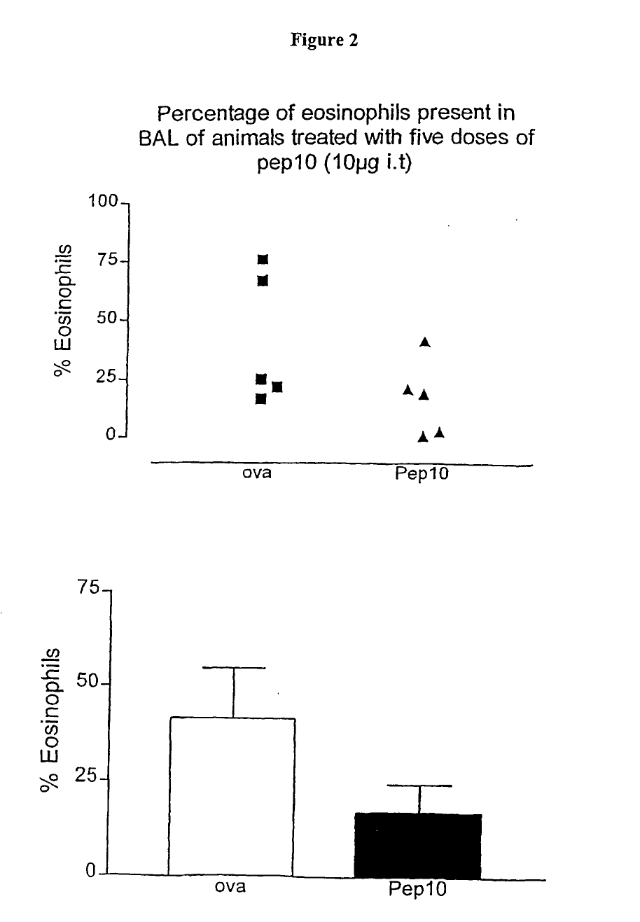

FIG. 2 shows the reduction in eosinophil levels in mice with ovalbumin-induced pulmonary eosinophilia after the administration of 5 doses of Mtcpn 10 (“Pep10”).

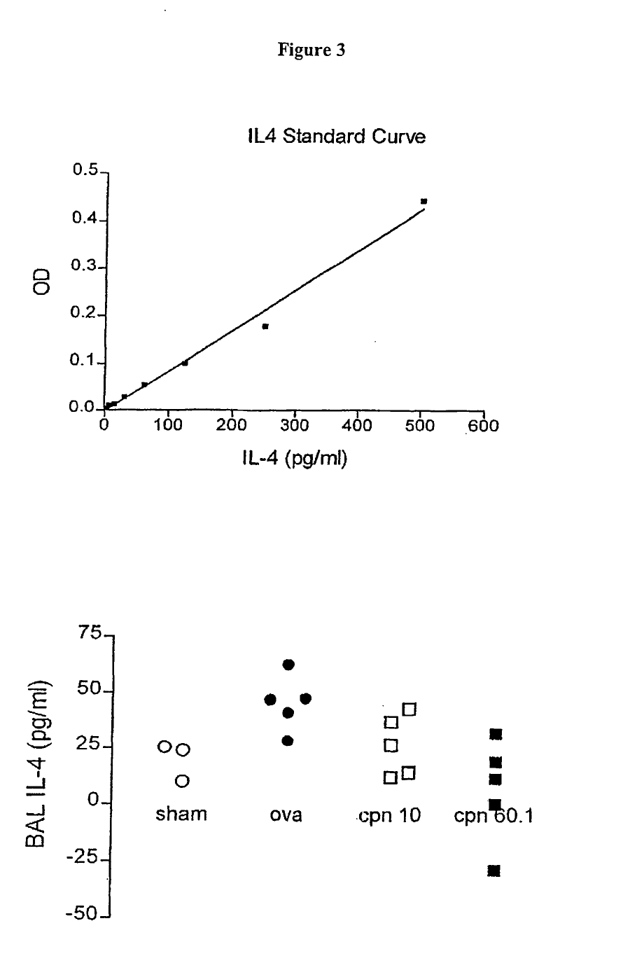

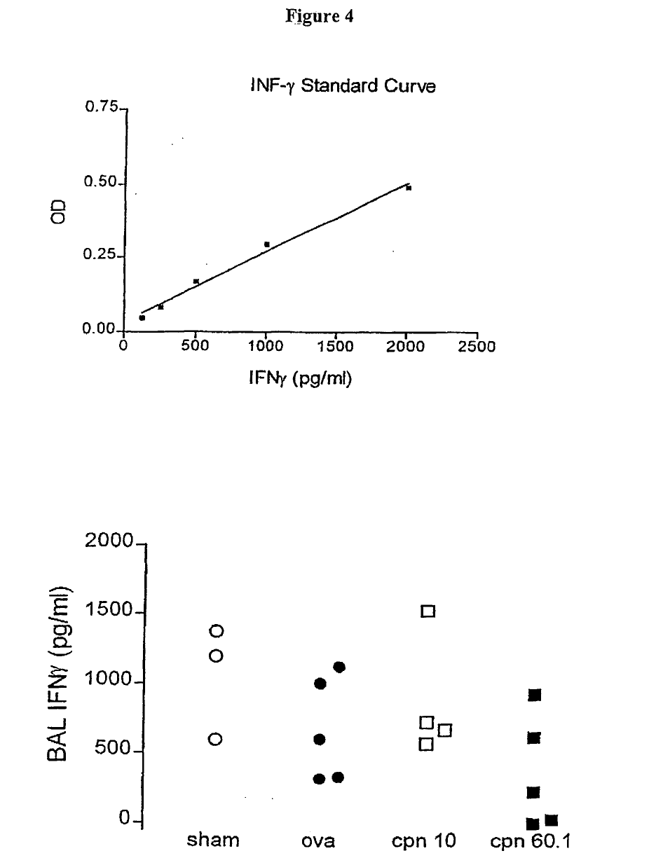

FIGS. 3 and 4 show the levels of IL4 (FIG. 3) and INF-γ (FIG. 4) detected in BALs.

Materials

Expression and purification of chaperonin 60 proteins M. tuberculosis cpn 10 was prepared by Prof M. Singh (WHO Collaborating Centre, Germany) using conventional chromatography as described below.

Purification of Recombinant cpn 10:

The 10 kDa antigen was expressed in a recombinant E. coli strain (IPTG-induced) as a fusion protein with maltose binding protein (MBP) using the commercially available pMAL-c vector (New England Biolabs). Initial purification was performed on an amylose affinity column. Afterwards the fusion protein was cleaved with factor Xₐ and the 10 kDa antigen was further purified by anion exchange chromatography, dialysed against 10 mM ammonium bicarbonate, aliquotted and lyophilized.

Great care was taken to check each batch of protein for LPS contamination using the Limulus assay (Tabona et al., 1998, J. Immunol., 161, p1414-1421). If LPS contamination was detected it was removed on a polymyxin B affinity column and levels of LPS re-assayed. Recombinant, LPS-low, chaperonins were further purified on a Reactive Red column to remove contaminating proteins and peptides (Tabona et al., 1998, supra).

The in vitro effects of cpn 10 on the production of IL-1β, IL-6, IL-8, IL-10, IL-12, TNFα and GM-CSF in human PBMCs was determined using 2-site ELISA as described by Tabona et al., 1998, supra.

### EXAMPLE 1

### Mycobacterium tuberculosis Cpn 10 Suppresses Asthma in the Mouse

This Example shows for the first time that in a murine model of allergic inflammation M. tuberculosis cpn 10 protein inhibited the recruitment of eosinophils to the airways in immunized mice. These data show that Mtcpn 10 modulates airway inflammation in the mouse and therefore, has important implications for allergic disease treatment and prevention.

Methods

Murine Model of Inflammation—A murine model of allergic inflammation that allows the quantitation of eosinophil and T lymphocyte recruitment in the airways following antigen challenge has been developed. Furthermore, a state of the art pulmonary monitoring system is used which allows changes in pulmonary mechanics to bronchoconstrictor agonists in vivo to be determined.

Immunisation Piotocol—C57B1/6 wild type (local supplier) 6-8 weeks old mice were immunised with ovalbumin (10 μg intraperitoneal injection; in 1 mg aluminium hydroxide) on day 0 and repeated 7 days later. On days 14, 15 and 16 mice were placed in a plexiglass container (12 L) and exposed to a nebulised solution of ovalbumin (10 mg/ml; De Vilibiss Ultraneb 90). Sham immunised wild type mice were injected with (1 mg aluminium hydroxide) on days 0 and 7 and also challenged with ovalbumin on days 14-16. Aerosol exposure was performed by exposure 3 times daily for 20 min at hourly intervals and bronchoalveolar lavage, collection of lungs for immunohistochemistry and lung mechanics was performed 24 h after the last aerosol challenge.

While ovalbumin is not a respiratory allergen often encountered by asthmatic subjects, the Th2 responses observed in murine models of inflammation are analogous to those observed following immunization with house dust mite. The Th2 cytokine profile generated by both allergens are similar. The advantage of using ovalbumin is that it is easily available and the specific activity of this allergen does not change between batches and therefore, we can control for antigen dose between batches.

BCG treatment: Six days following immunization with ovalbumin (10 μg), mice were injected with BCG (log BCG viable units: −4, −5 and −6) via the intravenous route. On day 7, mice received a booster injection with ovalbumin (10 μg). On day 13, mice received a second administration of BCG at the same dose and route as described for day 6. On day 14, mice were placed in a plexiglass box and exposed three times with nebulized ovalbumin (1% solution) for a period of 30 minutes at 1 hour intervals. This procedure was repeated on day 15 and 16. 24 hours after the last ovalbumin exposure, mice were anaesthetised and a bronchoalveolar lavage performed for the enumeration of eosinophils.

cpn 10 treatment: Mice from the same batch of animals were immunized to ovalbumin and treated with Mtcpn 10 (10 μg/animal) by direct instillation into the trachea. Mice were treated with Mtcpn 10 on day 6 and day 13 and then 30 min before the commencement of the challenge protocol on days 14, 15, and 16 (a total of 5 treatments).

Results

The results are shown in FIG. 2. There was significant suppression of the recruitment of eosinophils to the airways following ovalbumin challenge suggesting a protective effect for Mtcpn 10 (“Pep10”) in asthma.

Cytokine Measurements

We investigated the effect of cpn 10 over cytokine production in the serum and lavage collected 24 hours after the last challenge. We performed measurements of IL4, IL-5 and INF-γ levels in samples collected from groups of mice treated with 10 μg of cpn 10 and 56 μg of 60.1, both administered in a 5 doses scheme of treatment.

It was not possible to detect cytokine levels in the serum 24 hours after the last challenge and IL5 was not detected in any sample at this time. The levels of IL4 and INF-γ detected in BALs are shown in FIG. 3 and FIG. 4.

An important advantage of cpn 10 is that it can provide a prophylactic agent as distinct from an agent which is used to treat acute symptoms. In other words, cpn10 treatment can prevent allergic conditions such as asthma from developing.

The use of cpn 10 in the prevention and treatment of asthma is described in this example. Skilled persons will understand that the results obtained are relevant to other allergic conditions such as rhinitis/hay fever, eczema and anaphylaxis because all of the allergic conditions have a common mechanism (over-reactivity of Th-2 cells). Hence, if cpn10 can inhibit asthma, it should also inhibit the other allergic conditions.

These data demonstrate for the first time that Mtcpn 10 can suppress eosinophilic inflammation in a murine model of asthma. This show that this protein has the potential to modulate airways inflammation in the mouse, which has important implications for the treatment and prevention of allergic disease, autoimmune diseases and cancer.

1. A pharmaceutical composition comprising a nucleic acid molecule comprising
(i) the nucleotide sequence of FIG. 1, or
(ii) a sequence which has more than 66%, e.g. 70 or 75%, preferably more than 80%, e.g. more than 90 or 95% identity to sequence (i) or a sequence which hybridizes to sequence (i) under conditions of 2×SSC, 65° C. (wherein SCC=0.15M NaCl, 0.015M sodium citrate, pH 7.2) which encodes a functionally equivalent protein to the sequence encoded by the nucleotide sequence of FIG. 1, or (iii) a fragment of sequence (i) or (ii) encoding a functionally equivalent protein fragment; and a pharmaceutically acceptable excipient, diluent or carrier.

2. A pharmaceutical composition comprising a polypeptide comprising
(i) the amino acid sequence of FIG. 1, or
(ii) a sequence which has more than 60%, e.g. 65 or 70%, preferably more than 80%, e.g. more than 90 or 95% homology to sequence (i) which provides a functionally equivalent protein, or
(iii) a functionally equivalent fragment of any sequence (i) or (ii); and a pharmaceutically acceptable excipient, diluent or carrier.

3. A composition as claimed in claim 2 wherein the fragments are between 6 and 99 residues in length.

4. A composition as claimed in claim 3 wherein the fragment lengths are between 6 to 60.

5. A composition as claimed in claim 3 wherein the fragments are derived from or consisting of at least one of the following residues of the sequence shown in FIG. 1:
1-25
1-58
25-99
51-99
75-99

6. A pharmaceutical composition as claimed in any preceding claim for use in the manufacture of a medicament for the prevention and/or treatment of an allergic condition or cancer.

7. A pharmaceutical composition for use as claimed in claim 6 wherein the condition is selected from at least one of the following conditions:
eczema, dermatitis, allergic rhinitis, allergic conjunctivitis, allergic airway diseases, hyper-eosinophilic syndrome, contact dermatitis, food allergy, and respiratory diseases characterized by eosinophilic airway inflammation and airway hyperresponsiveness, such as allergic asthma, intrinsic asthma, allergic bronchopulmonary aspergillosis, eosinophilic pneumonia, allergic bronchitis bronchiectasis, occupational asthma, reactive airway disease syndrome, interstitial lung disease, hypereosinophilic syndrome or parasitic lung disease.

8. A pharmaceutical composition for use as claimed in claim 7 wherein the condition is asthma.

9. A method for treating and/or preventing an allergic or cancerous condition comprising administering a therapeutically or prophylactically effective dose, or plurality of doses, of a composition as defined in any one of claims 1 to 8.

10. A method of stimulating cytokine production in a cell wherein said method comprises administration of a nucleic acid molecule comprising
(i) the nucleotide sequence of FIG. 1, or
(ii) a sequence which has more than 66%, e.g. 70 or 75%, preferably more than 80%, e.g. more than 90 or 95% identity to sequence (i) or a sequence which hybridizes to sequence (i) under conditions of 2×SSC, 65° C. (wherein SCC=0.15M NaCl, 0.015M sodium citrate, pH 7.2) which encodes a functionally equivalent protein to the sequence encoded by the nucleotide sequence of FIG. 1, or (iii) a fragment of sequence (i) or (ii) encoding a functionally equivalent protein fragment; or
a polypeptide comprising
(i) the amino acid sequence of FIG. 1, or
(ii) a sequence which has more than 60%, e.g. 65 or 70%, preferably more than 80%, e.g. more than 90 or 95% homology to sequence (i) which provides a functionally equivalent protein, or
(iii) a functionally equivalent fragment of sequence (i) or (ii); to said cell.

11. A method as claimed in claim 10 wherein the cytokine production is increased at least 10-fold relative to normal levels.

12. A method as claimed in claim 10 or 11 wherein the cytokines are selected from at least one of the group consisting of IL-1β, IL-2, IL-6, IL-8, IL-10, IL-12, TNFα, interferon-γ and GM-CSF.

13. A method of assessing the presence or concentration of a polypeptide or peptide as defined in any preceding claim in a sample wherein said sample is applied to a cell and the level of production of one or more cytokines is measured and compared to the level of production of said one or more cytokines in a control sample wherein the increase over control levels provides a correlation to the presence or concentration of said polypeptide or peptide in said sample.


SEQUENCE LISTING (1 sequence)

SEQ ID NO: 1 - DNA, 303 bp, Mycobacterium tuberculosis
gtggcgaaggtgaacatcaagccactcgaggacaagattctcgtgcaggccaacgaggccgagaccacgaccgcgtccggtctggtcattcctgacaccgccaaggagaagccgcaggagggcaccgtcgttgccgtcggccctggccggtgggacgaggacggcgagaagcggatcccgctggacgttgcggagggtgacaccgtcatctacagcaagtacggcggcaccgagatcaagtacaacggcgaggaatacctgatcctgtcggcacgcgacgtgctggccgtcgtttccaagtag